(19) United States
(12) Patent Application Publication
(10) Pub. No.: US 2013/0150878 A1
(43) Pub. Date: Jun. 13, 2013

(54) HAND-HELD APPARATUS AND METHOD FOR REDUCING WRINKLES IN HUMAN SKIN

(71) Applicant: Susan Church, Newport Beach, CA (US)

(72) Inventor: Susan Church, Newport Beach, CA (US)

(21) Appl. No.: 13/314,508

(22) Filed: Dec. 8, 2011

(51) Int. Cl. A61B 17/34

(52) U.S. Cl. 606/189

(57) ABSTRACT

A hand-held apparatus and method by which to reduce the depth of a single wrinkle or a group of wrinkles in the skin of a human subject. The apparatus includes an elongated handle coupled to a removable roller. In one preferred embodiment, the roller is a disk having a plurality of needles projecting outwardly and arranged in a single row around the circumference of the roller. In another preferred embodiment, the roller is a cylinder having a plurality of outwardly-projecting needles arranged in alternating rows and columns. The needles are sized to prick the subject's skin when the roller is rolled over and pressed down against the skin area to be treated so as to cause collagen to be produced to fill in the wrinkle and thereby make the subject's skin look fuller and smoother.

### BACKGROUND OF THE INVENTION

1. Field of the Invention

This invention relates to a hand-held apparatus and a method for reducing the depth of a single wrinkle or a group of wrinkles in the skin of a human subject to make the treated skin area look fuller and feel smoother.

2. Background Art

Skin wrinkling and similar imperfections are commonly caused by age, drying of the skin, accident, illness, etc. Such imperfections can affect the aesthetic appearance of the skin and cause a loss of self confidence in the individual. Skin treatments including the use of lasers, chemicals, abrasion techniques, and the like have been tried by those wishing to rid themselves of their skin wrinkles. However, such treatments are typically performed by a healthcare worker and can be expensive. Moreover, certain treatments have, on occasion, caused damage to the individual's skin.

What is therefore desirable is an easy to operate and inexpensive apparatus that can be used either by the individual or by a trained treatment provider outside of a traditional medical facility and even at home in order to reduce the effects of skin wrinkles so that the individual's skin might look fuller and feel smoother.

### SUMMARY OF THE INVENTION

Briefly, and in general terms, disclosed herein are a hand-held apparatus and method for reducing one or more lines or wrinkles formed in a human subject's skin. The apparatus includes an elongated rigid handle, one end of which having a gap located between a pair of opposing roller supports. Positioned within the gap between the roller supports of the handle is a roller. A pin runs axially through the roller and the roller supports to establish an axle around which the roller can rotate. A fastener captures one end of the axle pin to prevent the unintended disconnection of the pin from the roller supports and a separation of the roller from the handle. However, by first removing the fastener, the axle pin and the roller can be removed from the handle of the apparatus for repair or replacement.

According to a first embodiment, the roller carried by the roller supports of the handle has a wheel or disk shape. A single continuous row of (e.g., surgical stainless steel) needles is disposed circumferentially around the disk-shaped roller. The needles arc embedded within and project radially from the roller by a sufficient distance so as to be adapted to treat (i.e., penetrate) a single line or wrinkle formed in the subject's skin. According to a second embodiment, the roller has a cylindrical shape. Continuous rows and columns of needles are disposed around the cylindrical roller to avoid gaps and maximize the surface area of the roller that is covered by needles. To this end, the needles are preferably arranged on the cylindrical roller so that the positions of the needles in one row and in one column alternate with the positions of the needles in prior and succeeding rows and columns. The needles are embedded within and project from the roller by a sufficient distance so as to be adapted to treat (i.e., penetrate) a group of lines or wrinkles at the same time.

With the user grasping the handle of the apparatus, the roller is positioned on the subject's skin to cover either a single wrinkle (if the roller is disk-shaped) or a group of wrinkles (if the roller is cylindrically shaped). Pushing and pressing forces are simultaneously applied to the handle to cause the roller to roll over and press down against the wrinkle or wrinkles. The needles which project from the roller prick the subject's skin to cause collagen to be produced and flow to the site of the needle penetrations. The collagen will plump up and fill in the wrinkle or wrinkles to reduce the depth thereof and make the subject's skin look fuller and smoother.

### BRIEF DESCRIPTION OF THE DRAWINGS

FIG. 1 is a perspective view of a hand-held apparatus for use in reducing lines or wrinkles in the skin of a subject according to a first preferred embodiment of this invention;

FIG. 2 is a side view of the hand-held apparatus of FIG. 1;

FIG. 3 is a top view of the hand-held apparatus of FIG. 1;

FIG. 4 is an exploded view of the hand-held apparatus of FIG. 1;

FIG. 5 illustrates the method for using the hand-held apparatus of FIG. 1 for treating a single line or wrinkle in the skin of the subject;

FIG. 6 is a perspective view of a hand-held apparatus being used for reducing lines or wrinkles in the skin of a subject according to a second preferred embodiment of this invention;

FIG. 7 is an enlarged detail of a roller of the hand-held apparatus taken from FIG. 6;

FIG. 8 shows an optional cluster of needles to be carried by the hand-held apparatus of FIGS. 1-4 or FIGS. 6 and 7 to prick the subject's skin during the treatment of the subject's skin lines or wrinkles; and

FIG. 9 is a top view of the needle cluster viewed along lines 9-9 of FIG. 8.

### DESCRIPTION OF THE PREFERRED EMBODIMENT

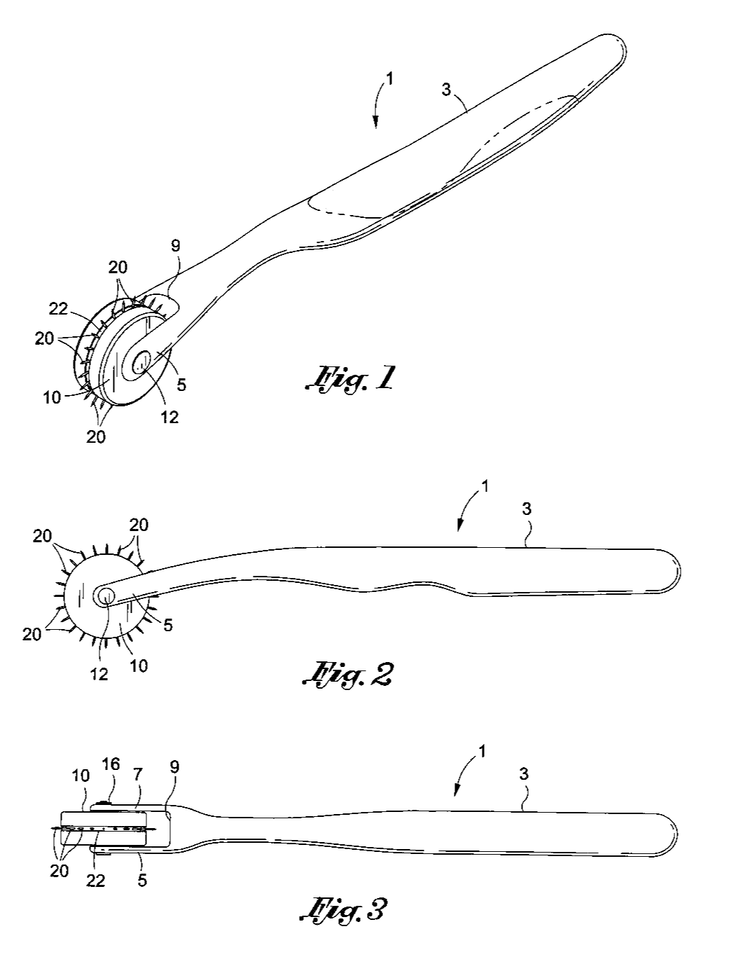

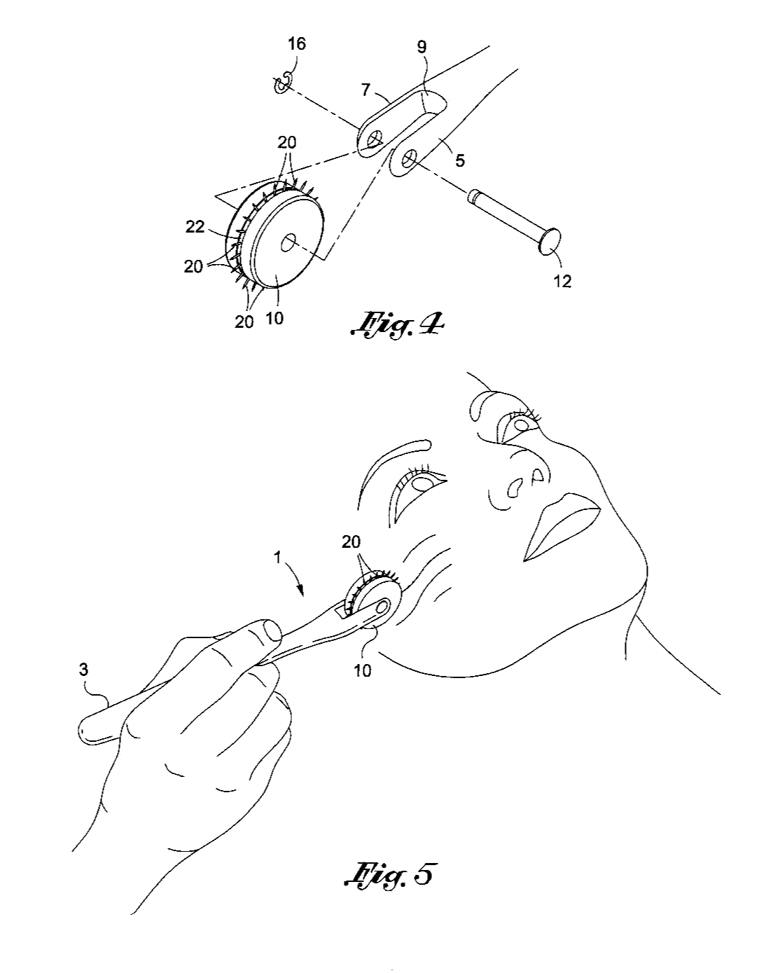

A hand-held apparatus and method according to a first preferred embodiment of the present invention for reducing the depth of a single targeted line or wrinkle in a human subject's skin are described while referring concurrently to FIGS. 1-4 of the drawings. The apparatus 1 includes an elongated handle 3 that is manufactured from a rigid material such as plastic, or the like, so as to be capable of withstanding a pressing force to be applied thereto for a purpose to be described hereinafter. The handle 3 is ideally about 12.0 cm long and about 1.0 cm wide and thick, although the precise dimensions of the handle are not to be considered a limitation of this invention. The handle 3 may be contoured (as shown) so as to facilitate a user grasping and manipulating the handle in his hand.

Located at one end of the handle 3 between a pair of opposing roller supports 5 and 7 is a gap 9. The gap 9 in handle 3 is sized to accommodate therewithin a roller 10. The roller 10 has a wheel or disk shape to enable the apparatus 1 to be used for the particular advantage of reducing the depth of and negative aesthetic appearance caused by a wrinkle formed in the skin of the subject. Accordingly, the roller 10 preferably has a diameter of approximately 1.75 cm and a width between supports 5 and 7 of about 7.0 mm so as to be able to cover and ride over a targeted wrinkle to be treated. Like the handle 3, the roller 10 is ideally manufactured from plastic, or the like.

A pin 12 runs axially through the disk-shaped roller 10 and the roller supports 5 and 7 of apparatus 1 to establish an axle around which the roller can rotate. A conventional fastener (e.g., a C-clip) 16 captures one end of the axle pin 12 to prevent an unintended disconnection of the pin from the roller supports 5 and 7 and a separation of the roller 10 from the handle 3. However, should it become necessary to remove the roller 10 for repair or replacement, the fastener 16 may be detached from the axle pin 12 so that the pin can be pulled outwradly through the roller supports 5 and 7 to enable the roller to be separated from the handle 3.

As an important feature of this embodiment, a set of needles 20 are embedded within and project radially from the roller 10. The needles 20 are evenly spaced from one another and located in a channel 22 that runs circumferentially around the roller 10. The needles 20 are preferably manufactured from surgical stainless steel, or the like. For the purpose of being able to pierce the subject's skin through a generally narrow wrinkle to be treated, a single row of needles 20 projects from the roller 10 at the channel 22 formed therearound to avoid excessive tissue trauma. In this same regard, the needles 20 project outwardly from the roller 10 by a distance of about 1.0 mm so as to be able to prick the subject's skin without inflicting unnecessary pain or injury. The channel 22 has a width of about 3.0 mm so that a typical skin wrinkle will fit therewithin as the roller 10 rolls thereover.

Turning now to FIG. 5 of the drawings, the method is described for using the apparatus 1 of FIGS. 1-4 to reduce a targeted wrinkle formed in the skin of the subject. With the user grasping the handle 3 of apparatus 1, the roller 10 is positioned on the subject's skin so as to lie upon the targeted wrinkle to be treated. Pushing and pressing forces are simultaneously applied by the user to the handle 3 by which to cause the roller 10 to press down against and roll over the wrinkle and the needles 20 which project from the roller to prick the subject's skin through the wrinkle. By virtue of the foregoing, collagen produced by the subject's body will flow to the sites of the penetrations made by the needles 20 so as to plump up and fill in the wrinkle. Hence, the depth of the wrinkle will be reduced so that the subject's skin will look fuller and feel smoother.

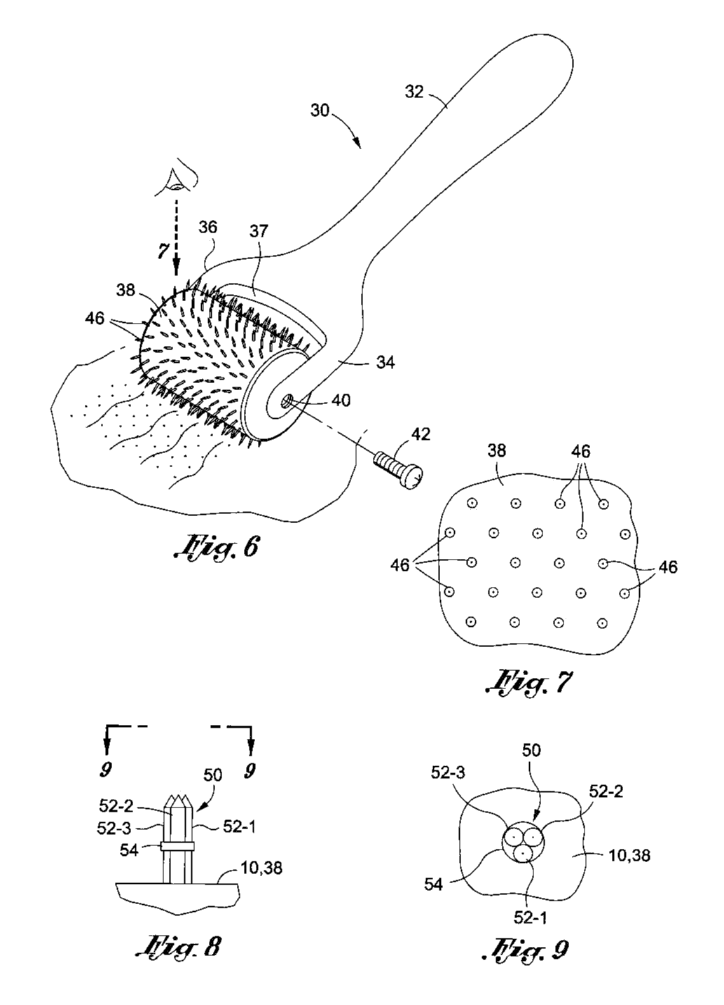

A hand-held apparatus and method according to a second preferred embodiment of the present invention are described while referring to FIGS. 6 and 7 of the drawings. While the apparatus 1 of FIGS. 1-4 is particularly suited for reducing the depth of a single line or wrinkle, the apparatus 30 of FIGS. 6 and 7 is suited to treat a wide area of the subject's skin and reduce the depth of a group of lines or wrinkles formed therein. Like the apparatus 1, the apparatus 30 has an elongated handle 32 that is manufactured from a rigid (e.g., plastic) material. The handle 32 is sized and shaped (i.e., contoured) to facilitate a user grasping and manipulating the handle in his hand.

Located at one end of the handle 32 between a pair of opposing roller supports 34 and 36 is a gap 37 which is sized to receive a (e.g., plastic) roller 38. The roller 38 has a cylindrical shape to enable the apparatus 30 to reduce the depth of and minimize the negative aesthetic appearance caused by wrinkles formed in the skin of the subject. In this case, however, the roller 38 is adapted to cover a large skin area and treat several wrinkles at the same time. Accordingly, the roller 38 preferably has a diameter of approximately 2.0 cm and a width between supports 34 and 36 of approximately 2.0 to 3.0 cm to be able to cover the wrinkles to be treated.

A pin 40 runs axially through the cylindrically-shaped roller 38 and the roller supports 34 and 36 of apparatus 30 to establish an axle around which the roller can rotate. One end of the axle pin 40 is headed (not shown), and the opposite end of pin 40 is hollow and threaded. A conventional fastener 42 having a threaded body and a wide head is rotated into mating engagement with the hollow threaded end of the axle pin 40 to prevent an unintended disconnection of the pin from the roller supports 34 and 36 and a separation of the roller 38 from the handle 32. However, should it become necessary to remove the roller 38 for repair or replacement, the fastener 42 may be detached from the axle pin 40 so that the pin can be pulled outwardly through the roller supports 34 and 36 to enable the roller to be separated from the handle 32.

As an important feature of this embodiment, a plurality of (e.g., surgical stainless steel) needles 46 are embedded within and project radially outward from the roller 38. The needles 46 are arranged in columns and rows around the roller 38 so as to be able to cover a relatively large surface area of the subject's skin. As best shown in FIG. 7, the needles 46 are arranged on the roller 38 so that positions of the needles in one row alternate with the positions of the needles in prior and succeeding rows across the roller 38. Likewise, the positions of the needles in one column alternate with the positions of the needles in prior and succeeding columns. The spacing between the needles 46 is determined by the size of the roller 38 so as to avoid gaps and maximize the surface area of the roller that is covered by needles projecting therefrom. Each needle 46 projects outwardly from the roller 38 by a distance of about 1.0 mm so as to be able to prick the subject's skin.

FIG. 6 illustrates the method for using the apparatus 30 to reduce wrinkles formed in the skin of the subject. With the user grasping the handle 32 of apparatus 30, the roller 38 is positioned on the subject's skin so as to lie over at least some of the wrinkles to be treated. Pushing and pressing forces are simultaneously applied by the user to the handle 32 by which to cause the roller 38 to press down against and roll over the wrinkles and the needles 46 which project from the roller to pierce the subject's skin through the wrinkles. As was previously explained, collagen produced by the subject's body will flow to the sites of the pricks made by the needles 46 so as to plump up and fill in the wrinkles so that the subject's skin will look fuller and feel smoother. The apparatus 1 may be moved from place-to-place over the subject's skin to treat different skin areas and/or different groups of wrinkles according to the method just described.

While the apparatus 1 and 30 herein disclosed are particularly useful for treating skin wrinkles, the apparatus can also be used to treat other skin imperfections such as pitted, depressed and scarred tissue areas. The apparatus 1 and 30 can be used by the subject himself or by a trained treatment provider. Since the rollers 10 and 38 may be replaced on an as-needed basis, the life of the apparatus can be advantageously maximized. What is more, the apparatus 1 and 30 may be used one after another to first treat a targeted wrinkle in the subject's skin and to then treat a wider surface area of the skin around the targeted wrinkle to better smooth the subject's skin adjacent the wrinkle.

Each of the needles (designated 20 and 46) that has been illustrated in FIGS. 1-7 consists of a single pointed tip located atop a single shaft to prick the subject's skin. In order to increase the size of the penetration and promote a more efficient release of collagen to the tissue area where the skin is pierced, a cluster of needles can be created in substitution of the aforementioned single needle. Referring in this regard to FIGS. 8 and 9 of the drawings, a cluster 50 is shown having three needles 52-1,52-2 and 52-3 lying side-by-side one another. A plurality of needle clusters 50 project from a roller (designated 10 and 38) in the manner and for the purpose earlier disclosed. The needles 52-1,52-2 and 52-3 of each cluster 50 are preferably bonded together by means of one or more solder joints 54. The combined needle tips of the needle cluster 50 create larger piercings through a wrinkle without having to manufacture thicker needles.

1. A hand-held apparatus for treating a depression formed in the skin of a subject, said apparatus including:
a handle to be grasped in the hand of a user;
a roller coupled to said handle, said roller being sized to extend across the depression to be treated; and
a plurality of needles projecting outwardly from said roller and having a length so as to pierce the depression formed in the subject's skin when said roller rolls over the depression in response to pushing and pressing forces simultaneously applied to said handle by the user, said roller comprising a disk having a diameter and a width, wherein said width is shorter than said diameter.

2. The hand-held apparatus recited in claim 1, wherein said plurality of needles are spaced from one another in a single row that runs continuously around the circumference of said roller disk.

3. The hand-held apparatus recited in claim 2, wherein there is a channel formed in said roller disk and extending circumferentially therearound, said single row of needles projecting from said roller disk through said channel.

4. The hand-held apparatus recited in claim 3, wherein the channel extending circumferentially around said roller disk has a width that is sized to receive therewithin the skin depression to be treated.

5. The hand-held apparatus recited in claim 1, wherein there is formed at one end of said handle a pair of opposing roller supports that are separated from one another by a gap, said roller disk located within the gap and coupled to said handle between said roller supports.

6. The hand-held apparatus recited in claim 5, further including a pin running axially through said roller disk and said pair of roller supports between which said roller disk is located such that said pin creates an axle around which said roller disk rotates when said roller disk rolls over the skin depression to be treated.

7. The hand-held apparatus recited in claim 6, further including a fastener connected to one end of said pin to prevent the removal of said pin from said roller disk and said pair of roller supports and thereby prevent a separation of said roller disk from said handle.

8. The hand-held apparatus recited in claim 7, wherein said fastener is detachable from the one end of said pin to enable said pin to be removed from said roller disk and said roller disk to be separated from said handle.

9. A hand-held apparatus for treating a group of depressions formed in the skin of a subject, said apparatus including:
a handle to be grasped in the hand of a user;
a roller coupled to said handle, said roller being sized to extend across at least some of the depressions to be treated; and
a plurality of needles projecting outwardly from said roller and having a length so as to pierce the at least some depressions formed in the subject's skin when said roller rolls over the depressions in response to pushing and pressing forces simultaneously applied to said handle by the user,
said roller comprising a cylinder having a diameter and a width, where said width is longer than said diameter.

10. The hand-held apparatus recited in claim 9, wherein said plurality of needles are arranged in an array of rows and columns around said cylindrical roller so that the positions of the needles in one row alternate with the positions of the needles in prior and succeeding rows, and the positions of the needles in one column alternate with the positions of the needles in prior and succeeding columns.

11. The hand-held apparatus recited in claim 9, wherein there is formed at one end of said handle a pair of opposing roller supports that are separated from one another by a gap, said cylindrical roller located within the gap and coupled to said handle between said roller supports.

12. The hand-held apparatus recited in claim 11, further including a pin running axially through said cylindrical roller and said pair of roller supports between which said cylindrical roller is located such that said pin creates an axle around which said cylindrical roller rotates when said cylindrical roller rolls over the depressions to be treated.

13. The hand-held apparatus recited in claim 12, further including a fastener connected to one end of said pin to prevent the removal of said pin from said cylindrical roller and said pair of roller supports and thereby prevent a separation of said cylindrical roller from said handle.

14. The hand-held apparatus recited in claim 13, wherein said fastener is detachable from the one end of said pin to enable said pin to be removed from said cylindrical roller and said cylindrical roller to be separated from said handle.

15. The hand-held apparatus recited in claim 9, wherein each of said plurality of needles comprises a cluster of needles bonded together and lying side-by-side one another, each needle of said cluster having a pointed tip lying atop a shaft.

16. A method for treating at least one depression formed in the skin of a subject, said method comprising the steps of:
locating a roller having a plurality of needles projecting therefrom across the depression to be treated;
pushing the roller over the depression so that at least some of the plurality of needles pierce the subject's skin through the depression to cause collagen to flow to and plump up the depression.

17. The method recited in claim 16, comprising the additional step of locating said plurality of needles on said roller so as to lie in a single row that runs continuously around the circumference of said roller.

18. The method recited in claim 16, comprising the additional step of locating said plurality of needles on said roller in an array of rows and columns around said roller so that the positions of the needles in one row alternate with the positions of the needles in prior and succeeding rows, and the positions of the needles in one column alternate with the positions of the needles in prior and succeeding columns.

19. The method recited in claim 16, comprising the additional steps of pushing said roller over the skin depression by applying a force to a handle coupled to said roller; and locating said roller between a pair of opposing roller supports that are spaced from each other and extend from one end of said handle.

20. The method recited in claim 19, comprising the additional steps of locating a removable pin through said roller and said pair of roller supports so that said roller rotates around said pin when said roller is pushed over the skin depression; and detachably connecting a fastener to at least one end of said pin to removably retain said removable pin in place through said roller and said roller supports such that said roller is removable from said roller supports when said pin is first removed from said roller and said roller supports.
